(19) United States
(12) Patent Application Publication
(10) Pub. No.: US 2002/0155439 A1
(43) Pub. Date: Oct. 24, 2002

(54) Method for generating a library of mutant oligonucleotides using the linear cyclic amplification reaction

(76) Inventors: Ana Rodriguez, Mundelein, IL (US); Volker Schellenberger, Palo Alto, CA (US); Huaming Wang, Fremont, CA (US)

Correspondence Address: Genencor International, Inc., 925 Page Mill Road, Palo Alto, CA, 94034, US

(21) Appl. No.: 09/729,520

(22) Filed: Dec. 4, 2000

(51) Int. Cl. C12Q 1/68; G01N 33/53; C12P 19/34; C12P 21/02

(52) U.S. Cl. 435/6; 435/7.1; 435/91.2; 435/69.1

(57) ABSTRACT

The present invention provides for a method of producing mutant nucleic acid molecules comprising preparing a first and second oligonucleotide corresponding to two different mutations in a template nucleic acid, mixing the oligonucleotides with a template to which they correspond so as to hybridize and subjecting the mixture to the linear cyclic amplification reaction. The present invention is particularly well suited for the development of libraries of mutant nucleic acids.

### BACKGROUND OF THE INVENTION

A. Field of the Invention

The present invention is related to the generation of libraries of mutant nucleic acid molecules from a precursor nucleic acid template or templates. The mutant library is then useful for selecting or screening purposes to obtain improved nucleic acid, protein or peptide product. More particularly, the present invention provides a novel method for the generation of combinatorial mutations.

B. Description of the State of the Art

Developing libraries of nucleic acids that comprise various combinations of several or many mutant or derivative sequences has recently been recognized as a powerful method of discovering novel products having improved or more desirable characteristics. A number of powerful methods for mutagenesis have been developed that when used iteratively with focused screening to enrich the useful mutants is known by the general term directed evolution.

For example, a variety of in vitro DNA recombination methods have been recently developed for the purpose of recombining more or less homologous nucleic acid sequences to obtain novel nucleic acids. For example, recombination methods have been developed comprising mixing a plurality of homologous, but different, nucleic acids, fragmenting the nucleic acids and recombining them using PCR to form chimeric molecules. For example, U.S. Pat. No. 5,605,793 discloses fragmentation of double stranded DNA molecules by DNase I. U.S. Pat. No. 5,965,408 discloses annealing of relatively short random primers to target genes and extending them with DNA polymerase. Each of these disclosures uses the polymerase chain reaction (PCR)-like thermocycling of fragments in the presence of DNA polymerase to recombine the fragments. Other methods have taken advantage of the phenomenon known as template switching, described in, e.g., Meyerhans, A., J.-P. Vartaanian and S. Wain-Hobson (1990) Nucleic Acids Res. 18,1687-1891. One shortcoming of these PCR based recombination methods however is that the recombination points tend to be limited to those areas of relatively significant homology. Accordingly, in recombining more diverse nucleic acids, the frequency of recombination is dramatically reduced and limited.

In many contexts, it is desirable to be able to develop libraries of mutant molecules that mix and match mutations which are known to be important or interesting due to functional or structural data. Several strategies toward combinatorial mutagenesis have been developed. In Stemmer et al., Biotechniques, vol. 18, no. 2 pp. 194-196(1995), the authors use a method they refer to as “gene shuffling” in combination with a mixture of specifically designed oligonucleotide primers to incorporate desired mutations into the shuffling scheme. Osuna et al., Gene, vol. 106, pp. 7-12(1991) designed an experiment in which synthetic DNA fragments comprising 50% wild type codon and 50% of an equimolar mixture of codons for each of the 20 amino acids at positions 144, 145 and 200 of EcoRI endonuclease. Tu et al., Biotechniques, vol. 20, no. 3, pp 352-353(1996) describes a method for generation of combination of mutations by using multiple mutagenic oligonucleotides which are incorporated into a mutagenic nucleotide by a single round of primer extension followed by ligation. Merino et al., Biotechniques, vol. 12, no. 4, pp. 508-509(1992) describes a method for single or combinatorial directed mutagenesis which utilizes a universal set of primers complementary to the areas that flank the cloning region of the pUC/M13 vectors used in the mutagenesis scheme for the purpose of optimizing yield of mutants.

In U.S. Pat. No. 5,923,419(Bauer et al.) a method for improved site-directed mutagenesis is described wherein the introduction of a mutation into circular DNA of interest is accomplished by means of mutagenic primer pairs that are selected so as to contain at least one mutation site with respect to the target DNA sequence, the primer pairs being at least partially complementary to each other and the mutation site being within the area of complementarity. The mutant DNA is then produced by extending the primer pairs against the template circular DNA using the linear cyclic amplification reaction.

While it is apparent that a number of methods exist, further and more efficient methods of producing libraries of mutant nucleic acids are desirable. For example, it would be desirable to be able to develop customized mutant nucleic acid libraries which have designed biases towards certain mutations. In addition, it would be desirable to be able to introduce contiguous and discontiguous mutations with the same degree of simplicity, current processes for discontiguous combinatorial mutation being particularly cumbersome. Further it would be desirable, in developing combinatorial mutation libraries, to reduce the level of unwanted mutation frequency, to achieve a high rate of mutational efficiency and to minimize and simplify the steps from primer design to expressed protein screening.

In the present invention, the inventors herein have determined a method for the combinatorial mutagenesis of nucleic acids which allows for optimization of the mutational scheme based on knowledge of the function and/or structure of the protein, while still developing a significant number of mutants with the potential for dramatically improved performance.

### SUMMARY OF THE INVENTION

According to the present invention, a method is provided for producing a library of mutant nucleic acid molecules comprising the steps of (a) obtaining a template nucleic acid; (b) preparing a first oligonucleotide corresponding to a first desired mutation within said template nucleic acid; (c) preparing a second oligonucleotide corresponding to a second desired mutation within said template nucleic acid; (d) mixing the oligonucleotides prepared in said steps (b) and (c) so as to hybridize said oligonucleotides to said template nucleic acid; (e) subjecting the mixture of step (d) to the linear cyclic amplification reaction to produce a library of mutant template nucleic acids. In a preferred method, the oligonucleotides in said steps (b) and (c) are discontiguous. In a further preferred embodiment, the first and second oligonucleotides are present in less than saturation concentration. In yet another preferred embodiment, the mixture of said step (d) further comprises non-mutagenic oligonucleotides corresponding to either or both of said first and second oligonucleotides.

In a further embodiment, the method of the invention further comprises the steps of: (f) transforming said mutant template nucleic acids from said library into a competent host cell; (g) expressing protein corresponding to said mutant nucleic acids in said host cell; (h) screening said expressed proteins for desired characteristics.

### DETAILED DESCRIPTION

The term “template nucleic acid” as used herein refers to a nucleic acid for which it is desired to develop a library of related nucleic acids the members of which have altered or modified characteristics compared to the template nucleic acid. Any source of nucleic acid, in purified or nonpurified form, can be utilized as the template nucleic acid or acids, provided it includes the specific nucleic acid sequence desired. Thus, the process may employ, for example, DNA or RNA, including messenger RNA, which DNA or RNA may be single stranded or double stranded. In addition, a DNA-RNA hybrid which contains one strand of each may be utilized. A mixture of any of these nucleic acids may also be employed, or the nucleic acids produced from a previous amplification reaction using the same or different primers may be so utilized. The specific nucleic acid sequence to be amplified may be only a fraction of a larger molecule or can be present initially as a discrete molecule, so that the specific sequence constitutes the entire nucleic acid. It is not necessary that the sequence to be amplified be present initially in a pure form; it may be a minor fraction of a complex mixture, such as a portion of the beta-globin gene contained in whole human DNA or a portion of nucleic acid sequence due to a particular microorganism which organism might constitute only a very minor fraction of a particular biological sample. The template nucleic acid may contain more than one desired specific nucleic acid sequence which may be the same or different. Therefore, the present process is useful not only for producing a library from one specific nucleic acid sequence, but also for creating variants simultaneously of more than one specific nucleic acid sequence located on the same or different nucleic acid molecules. The nucleic acid or acids may be obtained from any source, for example, from plasmids such as pBR322, from cloned DNA or RNA, or from natural DNA or RNA from any source, including bacteria, yeast, viruses, and higher organisms such as plants or animals. DNA or RNA may be extracted from blood, tissue material such as chorionic villi or amniotic cells by a variety of techniques such as that described by Maniatis et al, Molecular Cloning: A Laboratory Manual, (N.Y.: Cold Spring Harbor Laboratory, 1982), pp 280-281. Any specific nucleic acid sequence can be mutagenized by the present process. It is only necessary that a sufficient number of bases be known in sufficient detail so that at least two mutagenic oligonucleotide primers can be prepared which will hybridize to the desired sequence at desired positions along the sequence such that an extension product synthesized from one primer, when it is separated from its template (complement), can serve as a template for extension of the other primer into a nucleic acid of defined length. The greater the knowledge about the bases at the relevant portion of the sequence, the greater can be the specificity of the primers for the target nucleic acid sequence, and thus the greater the efficiency of the process.

The term “primer” as used herein refers to an oligonucleotide whether occurring naturally as in a purified restriction digest or produced synthetically, which is capable of acting as a point of initiation of synthesis when placed under conditions in which synthesis of a primer extension product which is complementary to a nucleic acid strand is induced, i.e., in the presence of nucleotides and an agent for polymerization such as DNA polymerase and at a suitable temperature and pH. The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the agent for polymerization. The exact lengths of the primers will depend on many factors, including temperature and source of primer. For example, depending on the complexity of the target sequence, the oligonucleotide primer typically contains 15-25 or more nucleotides, although it may contain fewer nucleotides. Short primer molecules generally require cooler temperatures to form sufficiently stable hybrid complexes with template.

The primers herein are selected to be “substantially” complementary to the different strands of each specific sequence to be amplified. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to be amplified to hybridize therewith and thereby form a template for synthesis of the extension product of the other primer.

The terms “mutagenic primer” or “mutagenic oligonucleotide” (used interchangeably herein) are intended to refer to oligonucleotide compositions which correspond to only a portion of the template sequence and which are capable of hybridizing thereto. With respect to mutagenic primers, the primer will not precisely match the template nucleic acid, the mismatch or mismatches in the primer being used to introduce the desired mutation into the nucleic acid library. As used herein, “non-mutagenic primer” or “non-mutagenic oligonucleotide” refers to oligonucleotide compositions which will match precisely to the template nucleic acid. In one embodiment of the invention, only mutagenic primers are used. In another preferred embodiment of the invention, the primers are designed so that for at least one region at which there is a desired mutagenic primer, there is also a non-mutagenic primer included in the oligonucleotide mixture which overlaps the mutagenic primer at least at the mutation site(s). By adding a mixture of mutagenic primers and non-mutagenic primers corresponding to at least one of said mutagenic primers, it is possible to produce a resulting nucleic acid library in which a variety of combinatorial mutational patterns are presented. For example, if it is desired that some of the members of the mutant nucleic acid library retain their precursor sequence at certain positions while other members are mutated at such sites, the non-mutagenic primers provide the ability to provide for a specific level of non-mutant members within the nucleic acid library for a given specific residue. The methods of the invention employ mutagenic and non-mutagenic oligonucleotides which are generally between 20-50 bases in length, more preferably about 25-45 bases in length. However, it may be desirable to use primers that are either longer than 20 bases or shorter than 50 bases so as to obtain the mutagenesis result desired. With respect to primer pairs, it is not necessary that the complementary oligonucleotides be of identical length. It is also not necessary that both mutagenic and non-mutagenic primers be used in the same amplification reaction.

Primers may be added in a pre-defined ratio according to the present invention. For example, if it is desired that the resulting library have a significant level of a certain specific mutation and a lesser amount of a different mutation at the same or different site, by adjusting the amount of primer added, it is possible to produce the desired biased library. Alternatively, by adding lesser or greater amounts of non-mutagenic primers, it is possible to adjust the frequency with which the corresponding mutation(s) are produced in the mutant nucleic acid library.

Several embodiments of the invention are possible with respect to the design of primers. For example, it is possible, and preferred in situations where it is desired to add more than 3 mutations, to use only one primer for each mutation. Where only two primers are used, depending on the intended transformation host, it may be desirable to use two complementary primers to ensure that reaction product is double stranded facilitating more efficient transformation. Similarly, by adding wildtype primer corresponding to the mutagenic primers at one or more mutation sites, it is possible to ensure that the combinatorial matrix represented in the mutant library includes wild type residues at the selected mutation sites.

The oligonucleotide primers may be prepared using any suitable method, such as, for example, the phosphotriester and phosphodiester methods or automated embodiments thereof. In one such automated embodiment diethylphosphoramidites are used as starting materials and may be synthesized as described by Beaucage et al, Tetrahedron Letters (1981), 22:1859-1862. One method for synthesizing oligonucleotides on a modified solid support is described in U.S. Pat. No. 4,458,055. It is also possible to use a primer which has been isolated from a biological source (such as a restriction endonuclease digest).

“Contiguous mutations” means mutations which are presented within the same oligonucleotide primer. For example, contiguous mutations may be adjacent or nearby each other, however, they will be introduced into the resulting mutant template nucleic acids by the same primer.

“Discontiguous mutations” means mutations which are presented in separate oligonucleotide primers. For example, discontiguous mutations will be introduced into the resulting mutant template nucleic acids by separately prepared oligonucleotide primers.

Controlling the concentration of mutagenic and corresponding non-mutagenic primers provides additional advantages to the invention. Specifically, using mutagenic or non-mutagenic oligonucleotides in relatively low concentrations compared to that used in conventional amplification techniques, i.e., at “a concentration less than saturation level” can result in varying frequencies of mutational combinations compared to standard techniques. By “saturation level”, Applicants mean that all of the mutagenic and corresponding non-mutagenic primers will be added in limiting quantities as compared to other reaction starting products. For purposes of comparison, consider that a typical PCR reaction, as described in Sambrook, J., E. F. Fritsch and T. Maniatis Molecular cloning: A Laboratory Manual, Vol. 2 pp. 14-18 [1989] describes 0.2 mM of each dNTP, resulting in a total concentration of dNTPs of 0.8 mM. Using this mixture to synthesize a product of I kb length requires 2000 moles of nucleotides to synthesize 1 mole of PCR product. Consequently, a reaction mixture containing 0.8 mM dNTPs can give a theoretical yield of 0.4 μM of PCR product. In practice, the yield will be substantially lower because a fraction of the dNTPs are hydrolyzed during the reaction and other side reactions will take up nucleotides. In addition other factors such as buffer capacity and enzyme activity limit the yield of an amplification reaction. In Sambrook, the author uses primers at concentrations of 1 μM. One of each primer molecules is thus required for the formation of one molecule of reaction product. Consequently, this concentration of primers leads to a theoretical yield of 1 μM of reaction product, a quantity which is substantially higher than the theoretical yield based on the concentration of dNTPs. Thus, a typical reaction involves the use of primers in significantly greater concentration in relation to the utilized dNTPs with a result that the primers are not completely used up during the reaction. While the linear cyclic amplification reaction differs from the PCR reaction in many ways, as described elsewhere herein, the effect of limiting primer concentration to facilitate masking hybridization efficiency differences is similar.

The optimal concentration of the mixture of primers with respect to dNTP and template concentrations will often depend on the specific reaction conditions but can be determined using routine experimentation well within the skill of the average technician in the field. For example, such optimal concentration may be determined experimentally by performing a series of parallel reactions using different concentrations of the primer mixture. Typically, the optimal primer concentration will be in a range such that product concentration is high enough to be detected by an agarose gel but that adding higher concentrations of primer mixture leads to higher concentrations of products, establishing that primer concentration is the limiting factor in the reaction. The present invention is not confined to absolute concentrations and variations are possible resulting from the specifics of the amplification reaction conditions and their effect on the component reagents in the reaction. Instead, in the present invention, a “less than saturation concentration” means that the oligonucleotide primers which are contributing to the combinatorial mutagenesis scheme are exhausted during the amplification reaction.

Any specific nucleic acid sequence can be mutagenized by the present process. It is only necessary that a sufficient number of bases be known in sufficient detail so that at least two mutagenic oligonucleotide primers can be prepared which will hybridize to the desired sequence at desired positions along the sequence such that an extension product synthesized from one primer, when it is separated from its template (complement), can serve as a template for extension of the other primer into a nucleic acid of defined length. The greater the knowledge about the bases at the relevant portion of the sequence, the greater can be the specificity of the primers for the target nucleic acid sequence, and thus the greater the efficiency of the process.

In the practice of the present invention, the linear cyclic amplification reaction is used to prepare a library of mutant nucleic acids. The term “linear cyclic amplification reaction” refers to a variety of enzyme mediated polynucleotide synthesis reactions that employ pairs of polynucleotide primers to linearly amplify a given polynucleotide and proceeds through one or more cycles, each cycle resulting in polynucleotide replication. Linear cyclic amplification reactions according to the present invention differ significantly from the polymerase chain reaction (PCR). The polymerase chain reaction produces an amplification product that grows exponentially in amount with respect to the number of cycles. Linear cyclic amplification reactions differ from PCR because the amount of amplification product produced in a linear cyclic amplification reaction is linear with respect to the number of cycles performed. A linear cyclic amplification reaction cycle typically comprises the steps of denaturing double-stranded template, annealing primers to the denatured template, and synthesizing polynucleotides from the primers. The cycle may be repeated several times so as to produce the desired amount of newly synthesized polynucleotide product. The linear cyclic amplification reaction is described in U.S. Pat. No. 5,923,419 (Bauer et al.), which is hereby incorporated by reference.

In general, the nucleic acid template is a DNA molecule and is in circular double stranded form. A plurality of mutagenic oligonucleotide pairs is prepared, wherein each oligonucleotide pair comprises at least a complementary section and the mutagenic oligonucleotides comprise within said complementary section at least one mismatch with the template nucleic acid molecule. The plurality of oligonucleotide pairs is annealed to the double stranded circular DNA template. The oligonucleotide primers may or may not be phosphorylated at the 5′ end. As the DNA molecule for mutagenesis is double stranded, the annealing step is generally preceded by a denaturation step. The annealing step is typically part of a cycle of a linear cyclic amplification reaction. After annealing of the oligonucleotide primer pairs, mutagenized DNA strands are synthesized from the mutagenic primers and the wild type primers retain the template DNA sequence. The linear cyclic amplification reaction may be repeated through several cycles until a sufficient variety of mutagenized nucleic acids are developed to produce a library. Typically, Applicants believe that it is desirable to repeat the reaction a number of times which equals the number of primers added, i.e., if 10 mutagenic primers are used, then in this preferred embodiment, 10 cycles should are performed. However, it is likewise useful to use less or greater numbers of cycles depending on the specific reaction, the library desired and efficient protocol requirements. Optionally, any remaining template strand can preferably be degraded by means known in the art, for example by endonuclease digestion, so that only mutagenized DNA remains in the mixture. The double stranded mutagenized circular DNA molecules which are produced are transformed into a suitable host cell. Transformed host cells may be isolated as colonies under conditions suitable for analyzing expressed protein product and/or nucleic acid product and screened for the desired protein or nucleic acid characteristic as appropriate.

In a preferred embodiment, non-mutagenic oligonucleotides are added which correspond with the mutagenic oligonucleotides with respect to the portion of the template nucleic acid to which they anneal.

It is also possible to use circular single stranded DNA by modifying the above procedure as follows. Instead of adding mutagenic oligonucleotide primer pairs, only one mutagenic primer and one non-mutagenic primer are added for each desired site for mutagenesis, the primers being complementary to the relevant template nucleic acid. After the primers are annealed to the template nucleic acid, synthesis of the mutagenic and non-mutagenic strands proceeds so as to produce double stranded circular DNA corresponding to both the mutant and the non-mutagenic form of the nucleic acid with respect to the mutations conferred by the particular primer pair.

An important advantage of the use of the present invention is the ease of the method with respect to producing clones from the library. For example, as opposed to PCR in which the relevant segments of amplified DNA must be separated, purified and ligated into an appropriate vector, it is possible using the present invention to directly produce circular DNA molecules suitable for tranformation directly into a competent host, i.e., without ligation.

Conditions which allow a primer to extend on a template generally include a polymerase, nucleotides and a suitable buffer. Polymerases for use in linear cyclic amplification reactions can be either thermostable or non-stable polymerase enzymes. Polymerases will not have the tendency to displace the primers that are annealed to the template, thereby producing mutagenized template nucleic acid. Preferably the polymerase used is a thermostable polymerase such as the pfu Turbo DNA polymerase (Stratagene), the Taq polymerase, phage T7 polymerase, phage T4 polymerase, DNA polymerase I and other known polymerases known in the art which are useful in primer extension. When the DNA molecule for mutagenesis is relatively long, such as entire operons or large genes, it is useful to use a mixture of thermostable DNA polymerases, wherein one of the DNA polymerases has 5′-3′ exonuclease activity and the other DNA polymerase lacks 5′-3′ exonuclease activity. A description of how to amplify long regions of DNA using these polymerase mixtures can be found in, among other places, U.S. Pat. No. 5,436,149.

In one embodiment, the products encoded by the nucleic acids generated according to the invention retain their function as in the protein encoded by the template nucleic acid, such as catalytic activity, but have an altered property with respect to some desired characteristic. A modified nucleic acid or protein as used herein refers to any sequence which has been manipulated to contain at least a portion of another molecule, ranging from at least one residue to as many as the entire sequence minus one residue.

Generally, the methods of the invention are useful for the generation of novel mutant nucleic acids. These novel nucleic acids may encode useful proteins, such as novel receptors, ligands, antibodies and enzymes. These novel nucleic acids may also comprise untranslated regions of genes, untranslated regions of genes, introns, exons, promoter regions, enhancer regions terminator regions, recognition sequences and other regulatory sequences for gene expression.

Thus, the methods of the invention provide for the formation of mutant nucleic acids ranging from 50-100 bp to several Mbp. The mutant nucleic acid library of the invention may be cloned, propagated and screened for a species or first subpopulation with a desired property. This results in the identification and isolation of, or enrichment for, a mutant nucleic acid encoding a polypeptide that has acquired a desired property.

The mutant nucleic acid library may be screened using assays for desired characteristics in the mutant nucleic acid or in the polypeptide encoded by the mutant nucleic acid.

As outlined above, the invention provides mutant nucleic acid libraries, wherein said nucleic acids encode polypeptides. The library of mutant nucleic acids will encode at least one polypeptide which has at least one property which is different from the same property of the corresponding template nucleic acid or corresponding precursor polypeptide. The properties described herein may also be referred to as biological activities.

The term “property” or grammatical equivalents thereof in the context of a polypeptide, as used herein, refers to any characteristic or attribute of a polypeptide that can be selected or detected. These properties include, but are not limited to oxidative stability, substrate specificity, catalytic activity, thermal stability, alkaline stability, pH activity profile, resistance to proteolytic degradation, Km, kcat, Kcat/km ratio, protein folding, inducing an immune response, ability to bind to a ligand, ability to bind to a receptor, ability to be secreted, ability to be displayed on the surface of a cell, ability to oligomerize, ability to signal, ability to be expressed, ability to stimulate cell proliferation, ability to inhibit cell proliferation, ability to induce apoptosis, ability to be modified by phosphorylation or glycosylation, ability to treat disease.

As used herein, the term “screening” has its usual meaning in the art and is, in general a multi-step process. In the first step, a mutant nucleic acid or variant polypeptide is provided. In the second step, a property of the mutant nucleic acid or variant polypeptide is determined. In the third step, the determined property is compared to a property of the corresponding naturally occurring nucleic acid, to the property of the corresponding naturally occurring polypeptide or to the property of the starting material (e.g., the initial sequence) for the generation of the mutant nucleic acid. The latter may also be a synthetic DNA.

It will be apparent to the skilled artisan that the screening for an altered property depends entirely upon the property of the starting material for the generation of the mutant nucleic acid. The skilled artisan will therefore appreciate that the invention is not limited to any specific property to be screened for and that the following description of properties lists illustrative examples only. Methods for screening for any particular property are generally described in the art. For example, one can measure binding, pH, specificity, etc., before and after mutation, wherein a change indicates an alteration. Preferably, the screens are performed in a high-throughput manner, including multiple samples being screened simultaneously, including, but not limited to assays utilizing chips, phage display, and multiple substrates and/or indicators.

A change in substrate specificity is defined as a difference between the kcat/Km ratio of the precursor protein and that of the variant thereof. The kcat/Km ratio is generally a measure of catalytic efficiency. Generally, the objective will be to generate variants of precursor proteins with a modified kcat/Km ratio for a given substrate when compared to that of the precursor protein, thereby enabling the use of the variant protein to more efficiently act on a target substrate or environment. However, it may be desirable to decrease efficiency. An increase in kcat/Km ratio for one substrate may be accompanied by a reduction in kcat/Km ratio for another substrate. This is a shift in substrate specificity and variants of precursor proteins exhibiting such shifts have utility where the precursor protein is undesirable, e.g., to prevent undesired hydrolysis of a particular substrate in an admixture of substrates. Km and kcat are measured in accordance with known procedures.

A change in oxidative stability is evidenced by at least about 10% or 20%, more preferably at least 50%, increase of enzyme activity when exposed to various oxidizing conditions. Such oxidizing conditions include, but are not limited to exposure of the protein to the organic oxidant diperdodecanoic acid (DPDA). Oxidative stability is measured by known procedures.

A change in alkaline stability is evidenced by at least about a 5% or greater increase or decrease (preferably increase) in the half life of the enzymatic activity of a variant of a precursor protein when compared to that of the precursor protein. In the case of e.g., subtilisins, alkaline stability can be measured as a function of autoproteolytic degradation of subtilisin at alkaline pH, e.g., 0.1 M sodium phosphate, pH 12 at 25° C. or 30° C. Generally, alkaline stability is measured by known procedures.

A change in thermal stability is evidenced by at least about a 5% or greater increase or decrease (preferably increase) in the half life of the catalytic activity of a variant of precursor protein when exposed to a relatively high temperature and neutral pH as compared to that of the precursor protein. In the case of e.g., subtilisins, thermal stability can be measured as a function of autoproteolytic degradation of subtilisin at elevated temperatures and neutral pH, e.g., 2 mM calcium chloride, 50 mM MOPS, pH 7.0 at 59° C. Generally, thermal stability is measured by known procedures.

A change in activity in pH buffer is evidenced by at least 5% or greater increase or decrease in higher or lower pH buffer activity on substrate of a variant of the precursor protein when compared to a precursor protein.

Receptor variants, for example are experimentally tested and validated in vivo and in vitro assays. Suitable assays include, but are not limited to, e.g., examining their binding affinity to natural ligands and to high affinity agonists and/or antagonists. In addition to cell-free biochemical affinity tests, quantitative comparisons are made comparing kinetic and equilibrium binding constants for the natural ligand to the naturally occurring receptor and to the receptor variants. The kinetic association rate (Kₒₙ) and dissociation rate (K_{off}) and the equilibrium binding constants (K_{d}) can be determined using surface plasmon resonance on a BIAcore instrument following the standard procedure in the literature [Pearce et al., Biochemistry 38:81-89(1999)]. For most receptors described herein, the binding constant between a natural ligand and its corresponding naturally occurring receptor is well documented in the literature. Comparisons with the corresponding naturally occurring receptors are made in order to evaluate the sensitivity and specificity of the receptor variants. Preferably, binding affinity to natural ligands and agonists is expected to increase relative to the naturally occurring receptor, while antagonist affinity should decrease. Receptor variants with higher affinity to antagonists relative to the non-naturally occurring receptors may also be generated by the methods of the invention.

Similarly, ligand variants, for example are experimentally tested and validated in vivo and in vitro assays. Suitable assays include, but are not limited to, e.g., examining their binding affinity to natural receptors and to high affinity agonists and/or antagonists. In addition to cell-free biochemical affinity tests, quantitative comparison are made comparing kinetic and equilibrium binding constants for the natural receptor to the naturally occurring ligand and to the ligand variants. The kinetic association rate (Kₒₙ) and dissociation rate (K_{off}), and the equilibrium binding constants (K_{d}) can be determined using surface plasmon resonance on a BIAcore instrument following the standard procedure in the literature [Pearce et al., Biochemistry 38:81-89(1999)]. For most ligands described herein, the binding constant between a natural receptor and its corresponding naturally occurring ligand is well documented in the literature. Comparisons with the corresponding naturally occurring ligands are made in order to evaluate the sensitivity and specificity of the ligand variants. Preferably, binding affinity to natural receptors and agonists is expected to increase relative to the naturally occurring ligand, while antagonist affinity should decrease. Ligand variants with higher affinity to antagonists relative to the non-naturally occurring ligands may also be generated by the methods of the invention.

By “protein” herein is meant at least two covalently attached amino acids, which may include proteins, polypeptides, oligopeptides and peptides. The protein may be a naturally occurring protein, a variant of a naturally occurring protein or a synthetic protein. The protein may be made up of naturally occurring amino acids and peptide bonds, or synthetic peptidomimetic structures, generally depending on the method of synthesis. Thus “amino acid”, in one embodiment, means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. Stereoisomers of the twenty conventional amino acids, unnatural amino acids such as α,α-disubstituted amino acids, N-alkyl amino acids, lactic acid, and other unconventional amino acids may also be suitable components for proteins of the present invention. Examples of unconventional amino acids include, but are not limited to: 4-hydroxyproline, γ-carboxyglutamate, ≮-N,N,N-trimethyllysine, ≮-N-acetyllysine, O-phosphoserine, N-acetylserine, N-formylmethionine, 3-methylhistidine, 5-hydroxylysine, ω-N-methylarginine, and other similar amino acids and imino acids. If non-naturally occurring side chains are used, non-amino acid substituents may be used, for example to prevent or retard in vivo degradations. Proteins including non-naturally occurring amino acids may be synthesized or in some cases, made by recombinant methods; see van Hest et al., FEBS Lett. 428:(1-2) 68-70(1998); and Tang et al., Abstr. Pap. Am. Chem. S218:U138-U138 Part 2 (1999), both of which are expressly incorporated by reference herein. Included within this definition are proteins whose amino acid sequence is altered by one or more amino acids when compared to the sequence of a naturally occurring protein.

A “variant protein” as used herein means a protein which is altered from a precursor protein. In the context of the present invention, this means that the nucleic acid template is modified, through the use of the presently described invention, in such a way that the protein expressed thereby is changed in terms of sequence. Thus, by using the present invention, a library of mutant nucleic acids is developed from the template nucleic acid(s) and this library is subsequently cloned and screened for expressed protein activities to detect useful variant proteins. Generally, this means that the protein has modified properties in some manner.

The nucleic acid templates may be from any number of eukaryotic or prokaryotic organisms or from archaebacteria. Suitable mammals include, but are not limited to, rodents (rats, mice, hamsters, guinea pigs, etc.), primates, farm animals (including sheep, goats, pigs, cows, horses, etc) and in the most preferred embodiment, from humans. Other suitable examples of eukaryotic organisms include plant cells, such as maize, rice, wheat, cotton, soybean, sugarcane, tobacco, and arabidopsis; fish, algae, yeast, such as Saccharomyces cerevisiae; Aspergillus and other filamentous fungi; and tissue culture cells from avian or mammalian origins. Suitable examples of prokaryotic organisms include gram negative organisms and gram positive organisms. Specifically included are enterobacteriaciae bacteria, pseudomonas, micrococcus, corynebacteria, bacillus, lactobacilli, streptomyces, and agrobacterium. Polynucleotides encoding proteins and enzymes isolated from extremophilic organisms, includining, but not limited to hyperthermophiles, psychrophiles, psychrotrophs, halophiles, barophiles and acidophiles, are also useful. Such enzymes may function at temperatures above 100° C. in terrestrial hot springs and deep sea thermal vents, at temperatures below 0° C. in arctic waters, in the saturated salt environment of the Dead Sea, at pH values at around 0 in coal deposits and geothermal sulfur-rich springs, or at pH values greater than 11 in sewage sludge.

The proteins can be intracellular proteins, extracellular proteins, secreted proteins, enzymes, ligands, receptors, antibodies or portions thereof.

The template nucleic acid encodes all or a portion of an enzyme. By “enzyme” herein is meant any of a group of proteins that catalyzes a chemical reaction. Enzymes include, but are not limited to (i) oxidoreductases; (ii) transferases, comprising transferase transferring one-carbon groups (e.g., methyltransferases, hydroxymethyl-, formyl-, and related transferases, carboxyl- and carbamoyltransferases, amidinotransferases) transferases transferring aldehydic or ketonic residues, acyltransferases (e.g., acyltransferases, aminoacyltransferas), glycosyltransferases (e.g., hexosyltransferases, pentosyltransferases), transferases transferring alkyl or related groups, transferases transferring nitrogenous groups (e.g., aminotransferases, oximinotransferases), transferases transferring phosphorus-containing groups (e.g., phosphotransferases, pyrophosphotransferases, nucleotidyltransferases), transferases transferring sulfur-containing groups (e.g., sulfurtransferases, sulfotransferases, CoA-transferases), (iii) Hydrolases comprising hydrolases acting on ester bonds (e.g., carboxylic ester hydrolases, thioester hydrolases, phosphoric monoester hydrolases, phosphoric diester hydrolases, triphosphoric monoester hydrolases, sulfuric ester hydrolases), hydrolases acting on glycosyl compounds (e.g., glycoside hydrolases, hydrolyzing N-glycosyl compounds, hydrolyzing S-glycosyl compound), hydrolases acting on ether bonds (e.g., thioether hydrolases), hydrolases acting on peptide bonds (e.g., α-aminoacyl-peptide hydrolases, peptidyl-amino acid hydrolases, dipeptide hydrolases, peptidyl-peptide hydrolases), hydrolases acting on C—N bonds other than peptide bonds, hydrolases acting on acid-anhydride bonds, hydrolases acting on C—C bonds, hydrolases acting on halide bonds, hydrolases acting on P—N bonds, (iv) lyases comprising carbon-carbon lyases (e.g., carboxy-lyases, aldehyde-lyases, ketoacid-lyases), carbon-oxygen lyases (e.g., hydro-lyases, other carbon-oxygen lyases), carbon-nitrogen lyases (e.g., ammonia-lyases, amidine-lyases), carbon-sulfur lyases, carbon-halide lyases, other lyases, (v) isomerases comprising racemases and epimerases, cis-trans isomerases, intramolecular oxidoreductases, intramolecular transferases, intramolecular lyases, other isomerases, (vi) ligases or synthetases comprising ligases or synthetases forming C—O bonds, forming C—S bonds, forming C—N bonds, forming C—C bonds.

Carbonyl hydrolases are useful and comprise enzymes that hydrolyze compounds comprising O═C—X bonds, wherein X is oxygen or nitrogen. They include hydrolases, e.g., lipases and peptide hydrolases, e.g., subtilisins or metalloproteases. Peptide hydrolases include α-aminoacylpeptide hydrolase, peptidylamino-acid hydrolase, acylamino hydrolase, serine carboxypeptidase, metallocarboxy-peptidase, thiol proteinase, carboxylproteinase and metalloproteinase. Serine, metallo, thiol and acid proteases are included, as well as endo and exo-proteases.

In another embodiment of the invention, the template nucleic acid encodes all or a portion of a receptor. By “receptor” or grammatical equivalents herein is meant a proteinaceous molecule that has an affinity for a ligand. Examples of receptors include, but are not limited to antibodies, cell membrane receptors, complex carbohydrates and glycoproteins, enzymes, and hormone receptors.

Cell-surface receptors appear to fall into two general classes: type 1 and type 2 receptors. Type 1 receptors have generally two identical subunits associated together, either covalently or otherwise. They are essentially preformed dimers, even in the absence of ligand. The type 1 receptors include the insulin receptor and the IGF (insulin like growth factor) receptor. The type-2 receptors, however, generally are in a monomeric form, and rely on binding of one ligand to each of two or more monomers, resulting in receptor oligomerization and receptor activation. Type-2 receptors include the growth hormone receptor, the leptin receptor, the LDL (low density lipoprotein) receptor, the GCSF (granulocyte colony stimulating factor) receptor, the interleukin receptors including IL-1, IL-2, IL-3, IL-4, IL-5, IL-6, IL-7, IL-8, IL-9, IL-11, IL-12, IL-13, IL-15, IL-17, etc., receptors, EGF (epidermal growth factor) receptor, EPO (erythropoietin) receptor, TPO (thrombopoietin) receptor, VEGF (vascular endothelial growth factor) receptor, PDGF (platelet derived growth factor; A chain and B chain) receptor, FGF (basic fibroblast growth factor) receptor, T-cell receptor, transferrin receptor, prolactin receptor, CNF (ciliary neurotrophic factor) receptor, TNF (tumor necrosis factor) receptor, Fas receptor, NGF (nerve growth factor) receptor, GM-CSF (granulocyte/macrophage colony stimulating factor) receptor, HGF (hepatocyte growth factor) receptor, LIF (leukemia inhibitory factor), TGFα/β(transforming growth factor α/β) receptor, MCP (monocyte chemoattractant protein) receptor and interferon receptors (α,βand γ). Further included are T cell receptors, MHC (major histocompatibility antigen) class I and class II receptors and receptors to the naturally occurring ligands, listed below.

In one embodiment of the invention, the template nucleic acid encodes all or a portion of a ligand. By “ligand” or grammatical equivalents herein is meant a proteinaceous molecule capable of binding to a receptor. Ligands include, but are not limited to cytokines IL-1ra, IL-1, IL-1a, IL-1b, IL-2, IL-3, IL-4, IL-5, IL-6, IL-8, IL-10, IFN-β, INFγ, IFN-α-2a; IFN-α-2B, TNF-α; CD40 ligand (chk), human obesity protein leptin, GCSF, BMP-7, CNF, GM-CSF, MCP-1, macrophage migration inhibitory factor, human glycosylation-inhibiting factor, human rantes, human macrophage inflammatory protein 1β, hGH, LIF, human melanoma growth stimulatory activity, neutrophil activating peptide-2, CC-chemokine MCP-3, platelet factor M2, neutrophil activating peptide 2, eotaxin, stromal cell-derived factor-1, insulin, IGF-I, IGF-II, TGF-β1, TGF-β2, TGF-β3, TGF-α, VEGF, acidic-FGF, basic-FGF, EGF, NGF, BDNF (brain derived neurotrophic factor), CNF, PDGF, HGF, GCDNF (glial cell-derived neurotrophic factor), EPO, other extracellular signaling moieties, including, but not limited to, hedgehog Sonic, hedgehog Desert, hedgehog Indian, hCG; coagulation factors including, but not limited to, TPA and Factor VIIa.

In one embodiment of the invention, the template nucleic acid encodes all or a portion of an antibody. The term “antibody” or grammatical equivalents, as used herein, refer to antibodies and antibody fragments that retain the ability to bind to the epitope that the intact antibody binds and include polyclonal antibodies, monoclonal antibodies, chimeric antibodies, anti-idiotype (anti-ID) antibodies. Preferably, the antibodies are monoclonal antibodies. Antibody fragments include, but are not limited to the complementarity-determining regions (CDRs), single-chain fragment variables (scfv), heavy chain variable region (VH), light chain variable region (VL).

Information with respect to nucleic acid sequences and amino acid sequences for enzymes, receptors, ligands, and antibodies is readily available from numerous publications and several data bases, such as the one from the National Center for Biotechnology Information (NCBI).

Variant proteins are identified from the nucleic acid libraries of the invention generally through screening. Such screening can be performed by cloning the nucleic acids from the library into suitable host cells. In practicing preferred embodiments of the invention, screening does not require the insertion of the mutant nucleic acids produced hereby into vectors as the circularized template DNA used is directly transformable. Thus, it is possible to clone the vectors embodying the mutant nucleic acids directly into a suitable host cell for expression of protein which can be assayed. A discussion follows which is pertinent to the development of cloned host cells which can be used for screening variant proteins for useful properties, or alternatively, for expressing a selected nucleic acid which is developed using the methods described herein and isolated as a preferred nucleic acid for producing desirable proteins.

The expression vectors of the invention may be either self-replicating extrachromosomal vectors or vectors which integrate into a host genome. Generally, these expression vectors include transcriptional and translational regulatory nucleic acid operably linked to the nucleic acid encoding the variant protein. The term “control sequence” or grammatical equivalents thereof, as used herein, refer to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize polyadenylation signals and enhancers. In one embodiment of the invention the control sequences are generated by using the methods described herein.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the nucleic acid sequences being linked are contiguous, and, in the case of a secretory leader, contiguous and in reading frame. However, enhancers do not have to be contiguous. Linking is accomplished by ligation at convenient restriction sites. If such sites do not exist, synthetic oligonucleotide adaptors, linkers or the recombination methods of the herein described invention, are used in accordance with conventional practice. The transcriptional and translational regulatory nucleic acid will generally be appropriate to the host cell used to express the fusion protein; for example, transcriptional and translational regulatory nucleic acid sequences from Aspergillus are preferably used to express the protein in Aspergillus. Numerous types of appropriate expression vectors, and suitable regulatory sequences are known in the art for a variety of host cells. In one embodiment of the invention the control sequences are operably linked to a another nucleic acid by using the methods described herein.

When a secretory sequence leads to a low level of secretion of a protein, a replacement of the secretory leader sequence is desired. In this embodiment, an unrelated secretory leader sequence is operably linked to a variant protein encoding nucleic acid leading to increased protein secretion. Thus, any secretory leader sequence resulting in enhanced secretion of protein is desired. Suitable secretory leader sequences that lead to the secretion of a protein are known in the art. In another preferred embodiment, a secretory leader sequence of a naturally occurring protein or a variant protein is removed by techniques known in the art and subsequent expression results in intracellular accumulation of the recombined protein.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences. Promoter sequences encode either constitutive or inducible promoters. The promoters may be either naturally occurring promoters or hybrid promoters. Hybrid promoters, which combine elements of more than one promoter, are also known in the art, and are useful in the present invention. In a preferred embodiment, the promoters are strong promoters, allowing high expression in cells, particularly in filamentous fungi such as Aspergillus, such as the glucoamylase gene promoter.

In addition, the expression vector may comprise additional elements. For example, the expression vector may have two replication systems, thus allowing it to be maintained in two organisms, for example in filamentous fungi cells for expression and in a prokaryotic host for cloning and amplification. Furthermore, for integrating expression vectors, the expression vector can be integrated randomly into the genome or contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art. In addition, in a preferred embodiment, the expression vector contains a selectable marker gene to allow the selection of transformed host cells. Selection genes are well known in the art and will vary with the host cell used.

The nucleic acids are introduced into the cells, either alone or in combination with an expression vector. By “introduced into ” or grammatical equivalents herein is meant that the nucleic acids enter the cells in a manner suitable for subsequent expression of the nucleic acid. The method of introduction is largely dictated by the targeted cell type, discussed below. Exemplary methods include PEG mediated protoplast transformation, CaPO₄ precipitation, liposome fusion, lipofectin®, electroporation, viral infection, etc. The nucleic acids may stably integrate into the genome of the host cell, or may exist either transiently or stably in the cytoplasm (i.e. through the use of traditional plasmids, utilizing standard regulatory sequences, selection markers, etc.).

Proteins derived from the mutant libraries of the present invention are produced by culturing a host cell transformed either with an expression vector containing nucleic acid encoding the protein or with the nucleic acid encoding the protein alone, under the appropriate conditions to induce or cause expression of the protein. The conditions appropriate for protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculovirus used in insect cell expression systems is a lytic virus, and thus harvest time selection can be crucial for product yield.

Appropriate host cells include yeast, bacteria, archaebacteria, fungi, and insect and animal cells, including mammalian cells. Of particular interest are Drosophila melangaster cells, Saccharomyces cerevisiae and other yeasts, E. coli, Bacillus, SF9 cells, C129 cells, 293 cells, Neurospora, Trichoderma, Aspergillus, Fusarium, Penicilliuma, Streptomyces, BHK, CHO, COS, Pichia pastoris, etc.

In one embodiment, the proteins are expressed in mammalian cells. Mammalian expression systems are also known in the art, and include retroviral systems. A mammalian promoter is any DNA sequence capable of binding mammalian RNA polymerase and initiating the downstream (3′) transcription of a coding sequence for the fusion protein into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenlytion signals include those derived form SV40.

The methods of introducing exogenous nucleic acid into mammalian hosts, as well as other hosts, are well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

As will be appreciated by those in the art, the type of mammalian cells used in the present invention can vary widely. Basically, any mammalian cells may be used, with mouse, rat, primate and human cells being particularly preferred, although as will be appreciated by those in the art, modifications of the system by pseudotyping allows all eukaryotic cells to be used, preferably higher eukaryotes. As is more fully described below, a screen can be set up such that the cells exhibit a selectable phenotype in the presence of a bioactive peptide. As is more fully described below, cell types implicated in a wide variety of disease conditions are particularly useful, so long as a suitable screen may be designed to allow the selection of cells that exhibit an altered phenotype as a consequence of the presence of a peptide within the cell.

Accordingly, suitable mammalian cell types include, but are not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell) , mast cells, eosinophils, vascular intimal cells, hepatocytes, leukocytes including mononuclear leukocytes, stem cells such as haemopoetic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Suitable cells also include known research cells, including, but not limited to, Jurkat T cells, NIH3T3 cells, CHO, COS, etc. See the ATCC cell line catalog, hereby expressly incorporated by reference.

In one embodiment, the cells may be additionally genetically engineered, that is, they contain exogenous nucleic acid other than the recombined nucleic acid of the invention.

In a preferred embodiment, the proteins are expressed in bacterial systems. Bacterial expression systems are well known in the art. A suitable bacterial promoter is any nucleic acid sequence capable of binding bacterial RNA polymerase and initiating the downstream (3′) transcription of the coding sequence of the protein into mRNA. A bacterial promoter has a transcription initiation region which is usually placed proximal to the 5′ end of the coding sequence. This transcription initiation region typically includes an RNA polymerase binding site and a transcription initiation site. Sequences encoding metabolic pathway enzymes provide particularly useful promoter sequences. Examples include promoter sequences derived from sugar metabolizing enzymes, such as galactose, lactose and maltose, and sequences derived from biosynthetic enzymes such as tryptophan. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription.

In addition to a functioning promoter sequence, an efficient ribosome binding site is desirable. In E. coli, the ribosome binding site is called the Shine-Delgarno (SD) sequence and includes an initiation codon and a sequence 3-9 nucleotides in length located 3-11 nucleotides upstream of the initiation codon.

The expression vector may also include a signal peptide sequence that provides for secretion of the expressed protein in bacteria. The signal sequence typically encodes a signal peptide comprised of hydrophobic amino acids, which direct the secretion of the protein from the cell, as is well known in the art. The protein is either secreted into the growth media (gram-positive bacteria) or into the periplasmic space, located between the inner and outer membrane of the cell (gram-negative bacteria). For expression in bacteria, usually bacterial secretory leader sequences, operably linked to the recombined nucleic acid, are preferred.

In a preferred embodiment, the proteins of the invention are expressed in bacteria and/or are displayed on the bacterial surface. Suitable bacterial expression and display systems are known in the art [Stahl and Uhlen, Trends Biotechnol. 15:185-92(1997); Georgiou et al., Nat. Biotechnol. 15:29-34(1997); Lu et al., Biotechnology 13:366-72(1995); Jung et al., Nat. Biotechnol. 16:576-80(1998)].

The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways.

These components are assembled into expression vectors. Expression vectors for bacteria are well known in the art, and include vectors for Bacillus subtilis, E. coli, Streptococcus cremoris, and Streptococcus lividans, among others.

The bacterial expression vectors are transformed into bacterial host cells using techniques well known in the art, such as calcium chloride treatment, electroporation, and others.

In one embodiment, proteins are produced in insect cells. Expression vectors for the transformation of insect cells, and in particular, baculovirus-based expression vectors, are well known in the art.

In another preferred embodiment, proteins are produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica. Preferred promoter sequences for expression in yeast include the inducible GAL1,10 promoter, the promoters from alcohol dehydrogenase, enolase, glucokinase, glucose-6-phosphate isomerase, glyceraldehyde-3-phosphate-dehydrogenase, hexokinase, phosphofructokinase, 3-phosphoglycerate mutase, pyruvate kinase, and the acid phosphatase gene. Yeast selectable markers include URA3, ADE2, HIS4, LEU2, TRP1, and ALG7, which confers resistance to tunicamycin; the neomycin phosphotransferase gene, which confers resistance to G418; and the CUP1 gene, which allows yeast to grow in the presence of copper ions.

In a preferred embodiment, the proteins of the invention are expressed in yeast and/or are displayed on the yeast surface. Suitable yeast expression and display systems are known in the art (Boder and Wittrup, Nat. Biotechnol. 15:553-7 (1997); Cho et al., J. Immunol. Methods 220:179-88 (1998); all of which are expressly incorporated by reference). Surface display in the ciliate Tetrahymena thermophila is described by Gaertig et al. Nat. Biotechnol. 17:462-465(1999), expressly incorporated by reference.

In one embodiment, proteins are produced in viruses and/or are displyed on the surface of the viruses. Expression vectors for protein expression in viruses and for display, are well known in the art and commercially available (see review by Felici et al., Biotechnol. Annu. Rev. 1:149-83(1995)). Examples include, but are not limited to M13(Lowman et al., (1991) Biochemistry 30:10832-10838(1991); Matthews and Wells, (1993) Science 260:1113-1117; Stratagene); fd (Krebber et al., (1995) FEBS Lett. 377:227-231); T7(Novagen, Inc.); T4(Jiang et al., Infect. Immun. 65:4770-7 (1997); lambda (Stolz et al., FEBS Lett. 440:213-7(1998)); tomato bushy stunt virus (Joelson et al., J. Gen. Virol. 78:1213-7(1997)); retroviruses (Buchholz et al., Nat. Biotechnol. 16:9514(1998)). All of the above references are expressly incorporated by reference.

In addition, the proteins of the invention may be further fused to other proteins, if desired, for example to increase expression or increase stability. Once made, the proteins may be covalently modified. One type of covalent modification includes reacting targeted amino acid residues of a protein with an organic derivatizing agent that is capable of reacting with selected side chains or the N- or C-terminal residues of a protein. Derivatization with bifunctional agents is useful, for instance, for crosslinking a protein to a water-insoluble support matrix or surface for use in the method for purifying anti-protein antibodies or screening assays, as is more fully described below. Commonly used crosslinking agents include, e.g., 1,1-bis(diazo-acetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the “-amino groups of lysine, arginine, and histidine side chains [T. E. Creighton, Proteins: Structure and Molecular Properties, W. H. Freeman & Co., San Francisco, pp. 79-86(1983)], acetylation of the N-terminal amine, and amidation of any C-terminal carboxyl group.

Another type of covalent modification of the protein included within the scope of this invention comprises altering the native glycosylation pattern of the variant protein or of the corresponding naturally occurring protein. “Altering the native glycosylation pattern” is intended for purposes herein to mean deleting one or more carbohydrate moieties found in a protein, and/or adding one or more glycosylation sites that are not present in the respective protein.

Addition of glycosylation sites to a protein may be accomplished by altering the amino acid sequence thereof. The alteration may be made, for example, by the addition of, or substitution by, one or more serine or threonine residues to the protein (for O-linked glycosylation sites). The amino acid sequence may optionally be altered through changes at the DNA level, particularly by mutating the DNA encoding the protein at preselected bases such that codons are generated that will translate into the desired amino acids.

Another means of increasing the number of carbohydrate moieties on the protein is by chemical or enzymatic coupling of glycosides to the polypeptide. Such methods are described in the art, e.g., in WO 87/05330, published Sep. 11, 1987 and in Aplin and Wriston, CRC Crit. Rev. Biochem., pp. 259-306(1981).

Removal of carbohydrate moieties present on the protein may be accomplished chemically or enzymatically or by mutational substitution of codons encoding for amino acid residues that serve as targets for glycosylation. Chemical deglycosylation techniques are known in the art and described, for instance, by Hakimuddin et al., Arch. Biochem. Biophys., 259:52 (1987) and by Edge et al., Anal. Biochem., 118:131(1981). Enzymatic cleavage of carbohydrate moieties on polypeptides can be achieved by the use of a variety of endo- and exo-glycosidases as described by Thotakura et al., Meth. Enzymol., 138:350(1987).

Another type of covalent modification of a protein comprises linking the protein to one of a variety of non-proteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

In a preferred embodiment, the protein is purified or isolated after expression. The proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the protein may be purified using a standard anti-library antibody column. Ultrafiltration and diafiltration techniques, in conjunction with protein concentration, are also useful. For general guidance in suitable purification techniques, see Scopes, R., Protein Purification, Springer-Verlag, N.Y. (1982). The degree of purification necessary will vary depending on the use of the protein. In some instances no purification may be necessary.

Alternatively, it is possible to isolate variant nucleic acids from a population by a variety of selection methods. These methods may involve enrichment of the nucleic acid itself or of the one or multiple proteins encoded by that nucleic acid. Selection can be based on a growth advantage that is conferred by a mutant nucleic acid or by one or multiple proteins encoded by that nucleic acid. Alternatively, selection can be based on binding of DNA or its encoded protein to a ligand of interest using display methods such as ribosomal or phage display which are well known in the art.

The following examples are intended to exemplify preferred embodiments of the invention and are not intended to be limiting of the invention in any way, the invention being defined by the claims.

### EXAMPLES

Method for Saturated Mutagenesis to Build Libraries:

The purpose of these experiments was to build libraries of mutants, each of which would produce an altered protein. The mutation(s) in the target gene nucleic acid (a phenol oxidase gene from the fungus Stachybotrys) were either consecutive or non-consecutive residues within the target gene and were generated using one primer (in part (a)) or multiple primers (in part (b)). The protocol provides for the substitution of consecutive or non-consecutive sites with all 20 possible amino acids and is exemplified herein with up to four different residues selected for substitution in the one-primer method and 7 different residues in the multiple primer method. The reactions were completed using restriction enzymes only for removal of the wildtype plasmid from the reaction product, and using no electrophoresis gels or ethidium bromide. The protocols have the advantage of producing a diverse library of readily transformable DNA from a single amplification reaction. PFU Turbo DNA Polymerase (Stratagene) was used for its ability to amplify the entire plasmid.

(A) One Primer Method

The following experiments illustrate an embodiment of the invention wherein a single primer is used to produce a combinatorial library of mutations which are in close proximity to each other and are consecutive or non-consecutive.

Single and multiple saturated mutagenesis reactions were carried out in a final volume of 5 μL (made with deionised water) containing 10x reaction buffer from Stratagene (200 mM Tris-HCl (pH 8.8), 20 mM MgSO₄, 100 mM KCl, 100 mM (NH₄)₂SO₄, 1% Triton® X-100 and 1 mg/mL nuclease-free BSA). The template DNA plasmid was 7 kB including the gene insertion. 130 ng of forward and/or complementary strand primers were used so that the template/primer ratio was set at 1:200. 1 μL of 10 mM PCR Nucleotide mix (Boehringer Mannheim) was added to the reaction and the reaction tubes were put on ice. 1 μL (2.5 units/μL) of PFU Turbo DNA Polymerase (Stratagene) was added to the reaction mix and the solution was overlaid with 30 μL mineral oil. The reaction tubes were put back on ice. The cycler was pre-heated to 95° C. and the reaction was initiated by heating the tubes for 35 seconds at 95° C. Subsequently, amplification was performed as follows: 35 seconds at 95° C./1 minute and 5 seconds at 55° C./15 minutes and 30 seconds at 68° C. This cycle was repeated 15 more times for a total of 16 cycles. The tubes were set at 4° C. until they were ready to be used for subsequent reactions. 1 μL of Dpn I enzyme (20 units/μL) (New England Biolabs) was added to the reaction and the tubes were incubated at 37° C. for 1 hour. Following incubation, additional 1 μL of Dpn I enzyme (20 units/μL) was added to the reaction and the tubes were again incubated at 37° C. for 1 hour. The reaction contents were then transformed into competent E. coli cells (Top 10, 1-shot cells from Invitrogen) using methods known in the art. For all reactions, the ratio of template to primer was always maintained at 1:200.

The experimental protocol in this example used primers that comprised 15 nucleotides on either side of the mutagenic codon(s). Thus, the sequence for a single amino acid saturation primer was 15nt-NNS-15nt; where N represents all four nucleotides (A, T, G or C) and S represents two nucleotides (G or C). The use of such primers allows for all twenty possible amino acids to be substituted in the desired site. The sequence for double amino acid saturation primers used was 15nt-NNS-NNS-15nt, which allows for all twenty possible amino acids to be substituted in each of two consecutive sites to generate a theoretical 400 possible variants. For triple amino acid mutations, primers were designed in a way that allows for all twenty possible amino acids to be substituted in each of three consecutive sites or three non-consecutive, but nearby sites covered by the same primer (15nt-NNS-NNS-NNS-15nt or 15nt-NNS-NNS-XXX-NNS-15nt, where XXX is part of the specific sequence) to generate a theoretical 8000 possible variants. For quadruple amino acid mutations, the primers used were as follows: 15nt-NNS-NNS-NNS-NNS-15nt or 15nt-NNS-NNS-XXX-NNS-NNS-15nt to generate a theoretical 160,000 possible variants.

Using these primers, libraries were generated from the target oxidase gene. The following examples show the specific sequences used in four separate reactions to generate the single and multiple mutants (only the forward primer sequence was given):

EXPERIMENT #1: Single amino acid saturation primer:
5′-3′ TAC CAT GAC CAT GCC NNS TCC ATC ACC GCC GAG
EXPERIMENT #2: Contiguous double amino acid saturation primer:
5′-3′ CAT GAC CAT GCC ATG NNS NNS ACC GCC GAG AAC GCC
EXPERIMENT #3: Contiguous triple amino acid saturation primer:
5′-3′ CAG GCT GCC CGC ATG NNS NNS NNS CAT GAC CAT GCC ATG
EXPERIMENT #4: Discontiguous quadruple amino acid saturation primer.
5′-3′ GGA GAG AAC ACC TCT NNS NNS AGC NNS NNS TTG CAC GGC TCT TTC

Using this protocol, single, double, triple and quadruple amino acid changes were made in the target gene.

Results were as follows:

EXPERIMENT #1: Sequence analysis of 10 randomly chosen transformants showed that 8 were mutants, with 6 different amino acid substitutions.

EXPERIMENT #2: Sequence analysis of 10 randomly chosen transformants showed that 9 were mutants with 9 different combinations of amino acid substitutions.

EXPERIMENT #3: Sequence analysis of 12 randomly chosen transformants showed that 9 were mutants with 9 different combinations of amino acid substitutions.

EXPERIMENT #4: Sequence analysis of 10 randomly chosen transformants showed that 10 were mutants with 10 different combinations of amino acid substitutions.

As can be seen from the results, the present method provides a robust and efficient manner of creating a focused but diverse mutational library from a precursor gene.

(B) Multiple Primer Method

The following experiments illustrate an embodiment of the present invention wherein separate mutations are distributed within a template nucleic acid in a combinatorial fashion using multiple site-directed mutagenesis primers in one amplification reaction.

All experiments involved the use of multiple primers. Reactions were carried out in a final volume of 50 μL (made with deionised water) containing 10x reaction buffer from Stratagene (200 mM Tris-HCl (pH 8.8), 20 mM MgSO₄, 100 mM KCl, 100 mM (NH₄)₂SO₄, 1% Triton® X-100 and 1 mg/mL nuclease-free BSA). The template DNA plasmid (pGAPT-DO104 B) was 7 kB including the gene insertion. 130 ng each of three primer sets (sequences shown later) were used. 1 μL of 10 mM PCR Nucleotide mix (Boehringer Mannheim) was added to the reaction and the reaction tubes were put on ice. 1 μL (2.5 units/μL) of PFU Turbo DNA Polymerase (Stratagene) was added to the reaction mix and the solution was overlaid with 30 μL mineral oil. The reaction tubes were put back on ice. The cycler was pre-heated to 95° C. and the reaction was initiated by heating the tubes for 35 seconds at 95° C. Subsequently, amplification was performed as follows: 35 seconds at 95° C./1 minute and 5 seconds at 55° C./15 minutes and 30 seconds at 68° C. This cycle was repeated 15 more times for a total of 16 cycles. The tubes were set at 4° C. until they were ready to be used for subsequent reactions. 1 μL of Dpn I enzyme (20 units/μL) (New England Biolabs) was added to the reaction and the tubes were incubated at 37° C. for 1 hour. Following incubation, additional 1 μL of Dpn I enzyme (20 units/μL) was added to the reaction and the tubes were again incubated at 37° C. for 1 hour. The reaction contents were then transformed into competent E. coli cells (Top 10, 1-shot cells from Invitrogen) using standard methods. For all reactions, the ratio of template to each primer was 1:200 in the starting reaction mixture.

The following primers were used which correspond to various mutations within the Stachybotrys sp. Oxidase B gene which was used as the template nucleic acid. The mutation corresponds to the underlined region of the primer.

(A) L48Y
5′-3′ CAG CTG AGT CCT CCC TAT GCC TTG TAC GAA GTG
(B) M188F
5′-3′ GCC GAG AAC GCC TAC TTC GGT CAG GCT GGT GTC
(C) F254M
5′-3′ GGT CAG CCT TGG CCT ATG CTC AAC GTG CAG CCG
(D) E348Q
5′-3′ CTC GGT GTT GAG CCT CAG TTT GAT AAC ACT GAC
(E) R423A
5′-3′ GAG AAC CGT CTG CTC GCC AAT GTG CCC CGC GAC
(F) R483A
5′-3′ CTG GCT CGT CGT GAG ACT GTC TAT GTT GAG GCC
(G) N550A
5′-3′ CTC GGA GAG TTC GAG GCT GGC TCG GGT GAC TTC

Three strategies for generating multiple combinations of mutations according to the present invention are illustrated below. Each strategy offers the possibility of modified nucleic acid libraries and provided different advantages. When providing a combinatorial library of 2-3 mutations, it is simple and efficient to add the mutagenic primer and its complementary strand for each mutation (see Experiment # 5). In contrast, for experiments using greater numbers of mutagenic primers (i.e., attempting to introduce more than 3 primers), the applicants found that it is preferred to alternate the orientation of each mutagenic primer and to not add both the mutagenic primer and a complementary primer for each mutation. By alternating and using the mutagenic primer for a first mutation followed by a complementary primer for a second mutation and then a mutagenic primer for a third mutation (see e.g., Experiment #'s 7, 8 and 9), etc . . . worked efficiently and prevented difficulties associated with mixing a large number of mutagenic primer and a corresponding complementary primer for each mutation. Of course, in light of the specification, it is apparent to the skilled worker that many variations may be developed related to the specifics of the primers and the steps used while remaining within the concept of the present invention.

1. Mutation primers plus complementary strands (EXPERIMENT #5).

2. Mutation primers, their complementary strands and their respective wild-type primers (EXPERIMENT #6).

3. Mutation primer without complementary strand (EXPERIMENT #7, #8 AND #9).

RESULTS:

EXPERIMENT #5 (Three Mutational Primer Experiment)—Primers A, C and G. Sequence analysis of 10 randomly chosen transformants showed that 5 of the mutants had all three mutations, 3 different variants had two mutations, and 2 different variants had one mutation.

EXPERIMENT #6 (Three Mutational Primer Experiment)—Primers A, C and G with their respective wild type primers. Sequence analysis of 7 randomly chosen transformants showed that 1 of the analyzed mutants had all three mutations, 1 had two mutations, 4 had one mutation (no bias) and 1 had no mutations.

EXPERIMENT #7 (Four Mutational Primer Experiment)—Primers A and D and the complementary strands of primers B and E. Sequence analysis of 10 randomly chosen transformants showed that 2 had three mutations, 2 with two mutations, 4 with one mutation and 2 with no mutations.

EXPERIMENT #8 (Six Mutational Primer Experiment)—Primers A, C, F and the complementary strands of primers B, E and G. Sequence analysis of 9 randomly chosen transformants showed that that 5 of the mutants had 2 mutations and 2 had 1 mutation and 2 had 5 mutations.

EXPERIMENT #9 (Seven Mutational Primer Experiment)—Primers A, C, E and G and the complementary strands of primers B, D and F. Sequence analysis of 15 randomly chosen transformants showed that 2 had five mutations, 1 had 4 mutations, 5 had 3 mutations, 1 had 2 mutations, 4 had 1 mutation and 2 had no mutations.

As can be seen from the data using limited sample sets, the present methods are effective in producing in a combinatorial fashion a random distribution of mutations. From these data, it is apparent that a larger sample set, i.e., a large combinatorial library, would comprise nucleic acids corresponding to many different combinations of mutation.

1. A method of producing a library of mutant nucleic acid molecules comprising:
(a) obtaining a template nucleic acid;
(b) preparing a first oligonucleotide corresponding to a first desired mutation within said template nucleic acid;
(c) preparing a second oligonucleotide corresponding to a second desired mutation within said template nucleic acid;
(d) mixing the oligonucleotides prepared in said steps (b) and (c) so as to hybridize said oligonucleotides to said template nucleic acid;
(e) subjecting the mixture of step (d) to the linear cyclic amplification reaction to produce a library of mutant template nucleic acids.

2. The method according to claim 1, wherein said oligonucleotides in said steps (b) and (c) are discontiguous.

3. The method according to claim 1, wherein said step first and second oligonucleotides are present in less than saturation concentration.

4. The method according to claim 1, wherein the mixture of said step (d) further comprises non-mutagenic oligonucleotides corresponding to either or both of said first and second oligonucleotides.

5. The method according to claim 1, wherein said template nucleic acid corresponds to a desired protein product.

6. The method according to claim 4, wherein said protein product comprises an enzyme, hormone, vaccine, peptide therapeutic or antibody.

7. The method according to claim 4, further comprising the steps of:
(f) transforming said mutant template nucleic acids from said library into a competent host cell;
(g) expressing protein corresponding to said mutant nucleic acids in said host cell;
(h) screening said expressed proteins for desired characteristics.
